(19) United States
(12) Patent Application Publication
(10) Pub. No.: US 2020/0292424 A1
(43) Pub. Date: Sep. 17, 2020

(54) COMPONENT CONCENTRATION MEASURING DEVICE AND COMPONENT CONCENTRATION MEASURING METHOD

(71) Applicant: HONDA MOTOR CO., LTD., Tokyo (JP)

(72) Inventors: Tomoaki Ohashi, Wako-shi (JP); Shigenobu Mitsuzawa, Wako-shi (JP)

(21) Appl. No.: 16/813,812

(22) Filed: Mar. 10, 2020

(30) Foreign Application Priority Data
Mar. 15, 2019 (JP) 2019-048142

(51) Int. Cl. G01N 1/38; G01N 33/487; G01N 27/416

(52) U.S. Cl. CPC G01N 1/38; G01N 27/416; G01N 33/48707

(57) ABSTRACT

A component concentration measuring device includes a liquid collecting unit configured to collect a main liquid including a measurement target component which is an object to be measured and discharge a mixed liquid obtained by mixing the main liquid with an auxiliary liquid supplied from an outside; and a first sensing unit including a first flow path positioned downstream of the liquid collecting unit and an in-mixed-liquid concentration measuring unit positioned in the first flow path and configured to measure a first measurement target component concentration which is the concentration of the measurement target component in the mixed liquid.

### CROSS-REFERENCE TO RELATED APPLICATION

Priority is claimed on Japanese Patent Application No. 2019-048142, filed Mar. 15, 2019, the contents of which are incorporated herein by reference.

### BACKGROUND

### Field of the Invention

The present invention relates to a component concentration measuring device and a component concentration measuring method.

### Background

There is a technology for measuring the concentration of a specific component in sweat (refer to Japanese Unexamined Patent Application, First Publication No. 2017-198577). In the related art, the concentration is measured by filling a flow path with sweat and supplying the sweat which has filled the flow path to a sensor configured to measure the concentration using the pressure of sweat vapor and the capillary phenomenon.

### SUMMARY

In the related art, old sweat may remain in the vicinity of a sensor in some cases. For this reason, in the related art, only an amount of newly generated sweat may not be able to be measured and the accuracy of analysis may be poor in some cases.

Also, in the related art, the flow path needs to be filled with sweat. Thus, when an amount of sweating is small, an amount of object to be measured cannot be measured until a sufficient amount of sweat is collected in some cases. For this reason, in the related art, it takes a long time to collect sweat, the real-time property may be impaired, and the accuracy of analysis may be poor in some cases.

Such problems have an influence on a technology for measuring an amount of a specific component in sweat as well as a technology for measuring the amount of components in a liquid discharged from a discharge source such as sap discharged from a plant.

An object of an aspect of the present invention is to provide a technology for preventing a decrease in measurement accuracy of an amount of components in a liquid discharged from a discharge source.

(1) An aspect of the present invention is a component concentration measuring device including: a liquid collecting unit configured to collect a main liquid including a measurement target component to be measured and discharge a mixed liquid obtained by mixing the main liquid with an auxiliary liquid supplied from an outside; and a first sensing unit including a first flow path positioned downstream of the liquid collecting unit and an in-mixed-liquid concentration measuring unit positioned in the first flow path and configured to measure a first measurement target component concentration which is the concentration of the measurement target component in the mixed liquid.

(2) The component concentration measuring device may include: a second sensing unit including a second flow path positioned downstream of the liquid collecting unit and a mixed liquid amount measurement unit configured to measure a mixed liquid amount which is an amount of the mixed liquid flowing through the second flow path.

(3) The component concentration measuring device may further include: a component concentration calculation unit configured to calculate a second measurement target component concentration which is the concentration of the measurement target component in the main liquid based on the first measurement target component concentration, the mixed liquid amount, and an auxiliary liquid amount which is an amount of the auxiliary liquid supplied from the outside.

(4) The component concentration measuring device may further include: an auxiliary fluid supply unit configured to supply the auxiliary liquid to the liquid collecting unit, wherein the auxiliary fluid supply unit may be configured to supply the auxiliary liquid to the first flow path after the auxiliary liquid has been supplied to the first flow path and then an inter-process time which is a predetermined time has elapsed, and the in-mixed-liquid concentration measuring unit may be configured to measure the concentration of the measurement target component in the mixed liquid including the auxiliary liquid supplied to the first flow path by the auxiliary fluid supply unit after the inter-process time has elapsed.

(5) In the component concentration measuring device, the liquid collecting unit and the first sensing unit may be laminated.

(6) In the component concentration measuring device, at least two of the liquid collecting unit, the first sensing unit, and the second sensing unit may be laminated.

(7) Another aspect of the present invention is a component concentration measuring method including: collecting a main liquid including a measurement target component to be measured and discharging a mixed liquid obtained by mixing the main liquid with an auxiliary liquid supplied from an outside; and measuring a first measurement target component concentration which is the concentration of the measurement target component in the mixed liquid.

According to (1) to (7) described above, an increase in time required for collecting sweat is prevented and the real-time property of measurement is not impaired. For this reason, it is possible to prevent a decrease in measurement accuracy caused by a time required for collecting sweat.

According to (4) described above, it is possible to measure the concentration of a component which is an object to be measured by reducing an influence of old sweat. For this reason, it is possible to prevent a decrease in measurement accuracy of an amount of component which is an object to be measured.

According to (5) and (6) described above, it is possible to prevent an area of the device from increasing and prevent an increase in size of the device.

### BRIEF DESCRIPTION OF THE DRAWINGS

FIG. 1 is a diagram showing an example of a functional constitution of a sweat measuring device in an embodiment.

FIG. 2 is a diagram showing an example of a functional constitution of a fluid supply unit in the embodiment.

FIG. 3 is a diagram showing an example of a cross section of a supplied unit in the embodiment.

FIG. 4 is a diagram showing an example of a constitution of an in-mixed-liquid concentration measuring unit in the embodiment.

FIG. 5 is a diagram showing a working electrode when viewed from a direction perpendicular to that of FIG. 4.

FIG. 6 is a diagram showing a counter electrode when viewed from a direction perpendicular to that of FIG. 4.

FIG. 7 is a top view of a sweat collecting unit in the embodiment.

FIG. 8 is a top view of a first sensing unit in the embodiment.

FIG. 9 is a top view of the sweat collecting unit and the first sensing unit which have been laminated in the embodiment.

FIG. 10 is a flowchart for describing a flow of a process from when a start condition is satisfied to when the sweat measuring device calculates the concentration of a component to be measured in sweat in the embodiment.

### DESCRIPTION OF EMBODIMENTS

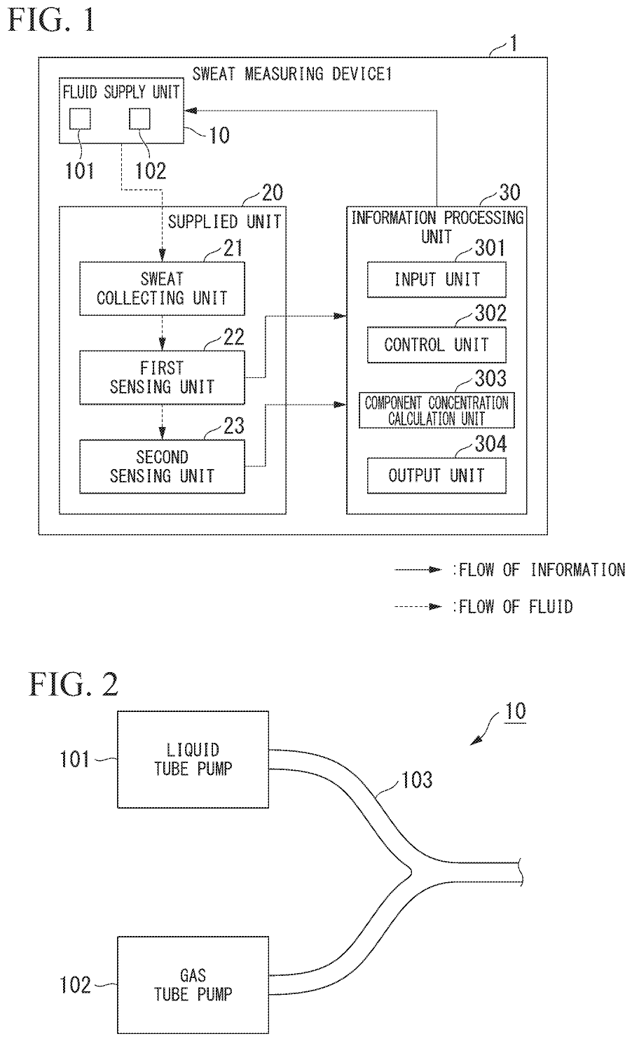

FIG. 1 is a diagram showing an example of a functional constitution of a sweat measuring device 1 in an embodiment. The sweat measuring device 1 measures an amount of component which is an object to be measured (hereinafter referred to as a “component to be measured”) contained in sweat. The component to be measured may be any component as long as it is a component contained in sweat. The component to be measured may be, for example, lactic acid, glucose, sodium, or potassium.

The sweat measuring device 1 includes a central processing unit (CPU), a memory, an auxiliary storage device, and the like connected through a bus and executes a program. The sweat measuring device 1 functions as a device which includes a fluid supply unit 10, a supplied unit 20, and an information processing unit 30 by executing a program.

The fluid supply unit 10 is connected to the supplied unit 20 using a tube and supplies an auxiliary liquid to the supplied unit 20.

The auxiliary liquid may be any liquid as long as it does not contain the component to be measured and does not cause a chemical reaction with the component to be measured. For example, when the component to be measured is lactic acid in sweat, the auxiliary liquid may be pure water.

FIG. 2 is a diagram showing an example of a functional constitution of the fluid supply unit 10 in the embodiment.

The fluid supply unit 10 includes a liquid tube pump 101, a gas tube pump 102, and a tube 103.

The liquid tube pump 101 is connected to the supplied unit 20 through the tube 103. The liquid tube pump 101 discharges the auxiliary liquid into the tube 103.

The gas tube pump 102 is connected to the supplied unit 20 through the tube 103. The gas tube pump 102 discharges an auxiliary gas into the tube 103. The auxiliary gas may be any gas as long as it does not contain the component to be measured and does not cause a chemical reaction with the component to be measured. For example, when the component to be measured is lactic acid in sweat, the auxiliary gas may be helium.

The fluid supply unit 10 sweeps a predetermined amount of the auxiliary liquid into the tube 103 using the liquid tube pump 101 and then transfers the auxiliary liquid to the supplied unit 20 using a gas pressure of the auxiliary gas discharged by the gas tube pump 102. In this way, the auxiliary liquid flows into the supplied unit 20.

A description will be provided with reference to FIG. 1 again.

The supplied unit 20 includes a sweat collecting unit 21, the first sensing unit 22, and a second sensing unit 23 and acquires various values associated with the auxiliary liquid and the component to be measured.

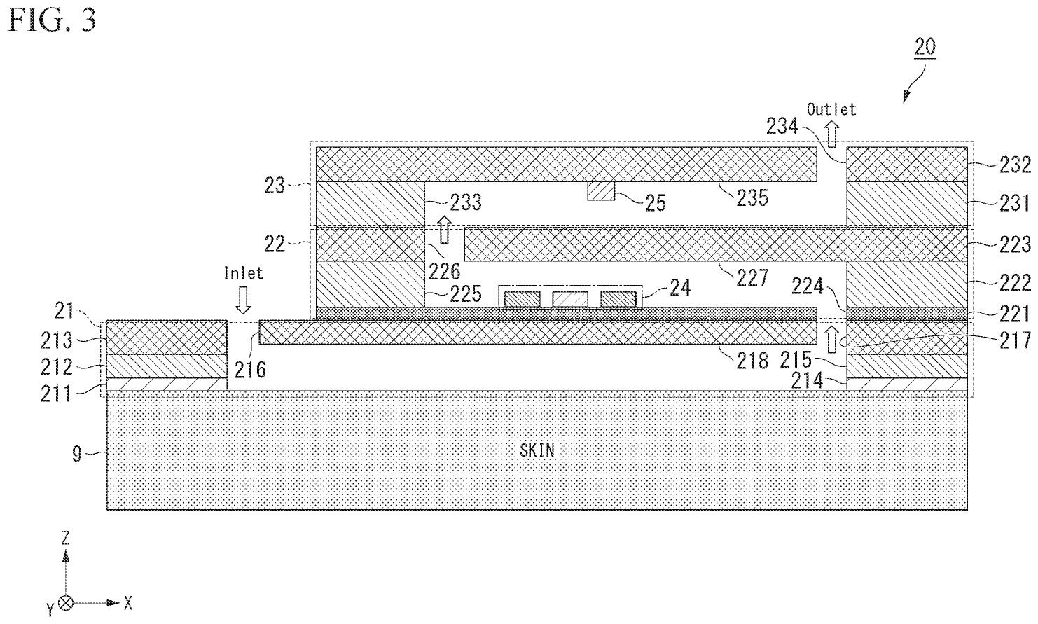

FIG. 3 is a diagram showing an example of a cross section of the supplied unit 20 in the embodiment.

FIG. 3 shows the sweat collecting unit 21, the first sensing unit 22, and the second sensing unit 23 laminated in this order in a direction perpendicular to a surface of skin 9. The skin 9 is an example of a discharge source of the component to be measured. The direction perpendicular to the surface of the skin 9 is, for example, a direction from an inside of the skin 9 to an outside of the skin and is a forward direction of a Z axis in FIG. 3. Hereinafter, a plane perpendicular to the Z axis is referred to as an XY plane.

The sweat collecting unit 21 includes a sheet-like first adhesive layer 211, a sheet-like second adhesive layer 212, and a sheet-like the first dielectric layer 213 laminated in this order in the forward direction of the Z axis.

The first adhesive layer 211 is in contact with the skin. The first adhesive layer 211 has an opening portion 214 formed in a surface thereof in contact with the skin.

The first adhesive layer 211 may be any type as long as it is the form of a sheet which adheres to the skin. The first adhesive layer 211 is, for example, a medical tape. A thickness (a length in a Z-axis direction) of the first adhesive layer 211 can be preferably 10 to 200 μm and more preferably 90 μm.

The opening portion 215 whose shape and size in the XY plane are substantially the same as the opening portion 214 is formed in the second adhesive layer 212. The opening portion 215 communicates with the opening portion 214.

The second adhesive layer 212 adheres the first adhesive layer 211 to the first dielectric layer 213. The second adhesive layer 212 may be any type as long as it is in the form of a sheet which adheres the first adhesive layer 211 to the first dielectric layer 213. The second adhesive layer 212 is, for example, a double-sided tape.

The thickness (a length in the Z-axis direction) of the second adhesive layer 212 can be preferably 100 to 500 μm and more preferably 200 μm.

The first dielectric layer 213 has two opening portions formed therein. Hereinafter, one of the opening portions is referred to as an opening portion 216. Hereinafter, the other of the opening portions is referred to as an opening portion 217. Sizes of the opening portion 216 and the opening portion 217 in the XY plane are smaller than sizes of the opening portion 214 and the opening portion 215 in the XY plane. The opening portion 216 communicates with the opening portion 214 and the opening portion 215. The opening portion 217 communicates with the opening portion 214 and the opening portion 215.

The first dielectric layer 213 is formed of a dielectric. The first dielectric layer 213 may be any type as long as it is a sheet-like dielectric. The first dielectric layer 213 may be, for example, a silicon sheet. A thickness (a length in the Z-axis direction) of the first dielectric layer 213 is preferably 100 to 1000 μm and more preferably 500 μm.

A flow path 218 is formed using the opening portion 214, the opening portion 215, the opening portion 216, and the opening portion 217. Since the flow path 218 is formed using the opening portion 214, the opening portion 215, the opening portion 216, and the opening portion 217, the opening portions are positioned in a surface in contact with the skin 9. For this reason, sweat discharged from the skin 9 flows into the flow path 218.

The auxiliary liquid flows into the sweat collecting unit 21 through the opening portion 216. The auxiliary liquid which has flowed into the opening portion 216 flows through the flow path 218. The auxiliary liquid flowing through the flow path 218 mixes with the sweat. Hereinafter, the auxiliary liquid which has mixed with the sweat is referred to as a mixed liquid. The mixed liquid is discharged through the opening portion 217.

In this way, the sweat collecting unit 21 collects the sweat containing the component to be measured which is an object to be measured and causes the mixed liquid obtained by mixing the sweat with the auxiliary liquid supplied from the fluid supply unit 10 positioned outside of the sweat collecting unit 21 to be discharged.

When thicknesses of the first adhesive layer 211, the second adhesive layer 212, and the first dielectric layer 213 are equal to or more than preferred lower limit values, it is possible to stabilize an output of the sweat measuring device 1. Furthermore, when thicknesses of the first adhesive layer 211, the second adhesive layer 212, and the first dielectric layer 213 are equal to or less than preferred upper limit values, it is easy to secure the flexibility of the supplied unit 20 and the supplied unit 20 easily follows the movement of the skin 9.

A description will be provided with reference to FIG. 3 again. The first sensing unit 22 includes a sheet-like first insulating layer 221, a sheet-like third adhesive layer 222, and a sheet-like second dielectric layer 223 laminated in this order in the forward direction of the Z axis.

The first insulating layer 221 is in contact with the first dielectric layer 213. The first insulating layer 221 has the opening portion 224 formed in a surface thereof in contact with the first dielectric layer 213. The opening portion 224 communicates with the opening portion 217.

The first insulating layer 221 is formed of an insulator. The first insulating layer 221 may be any type as long as it is a sheet-like insulator. The first adhesive layer 211 is, for example, a Kapton tape. A thickness (a length in the Z-axis direction) of the first insulating layer 221 can be preferably 10 to 100 μm and more preferably 50 μm.

In the third adhesive layer 222, the opening portion 225 having a size in the XY plane larger than that of the opening portion 224 is formed. The opening portion 225 communicates with the opening portion 224.

The third adhesive layer 222 adheres the first insulating layer 221 to the second dielectric layer 223. The third adhesive layer 222 may be any type as long as it is in the form of a sheet which adheres the first insulating layer 221 to the second dielectric layer 223. The third adhesive layer 222 may be, for example, a double-sided tape.

A thickness (a length in the Z-axis direction) of the third adhesive layer 222 can be preferably 200 to 700 μm and more preferably 580 μm.

In the second dielectric layer 223, the opening portion 226 having a size in the XY plane smaller than that of the opening portion 225 is formed. The opening portion 226 communicates with the opening portion 225. A line passing through a center of the opening portion 226 in the XY plane and parallel to the Z axis does not intersect with a line passing through a center of the opening portion 224 in the XY plane and parallel to the Z axis.

The second dielectric layer 223 is formed of a dielectric. The second dielectric layer 223 may be any type as long as it is a sheet-like dielectric. The second dielectric layer 223 may be, for example, a silicon sheet. A thickness (a length in the Z-axis direction) of the second dielectric layer 223 can be preferably 100 to 1000 μm and more preferably 500 μm.

A flow path 227 is formed using the opening portion 224, the opening portion 225, and the opening portion 226. The mixed liquid which has flowed through the flow path 218 flows into the first sensing unit 22 through the opening portion 224.

The mixed liquid which has flowed into the opening portion 224 flows through the flow path 227. The mixed liquid flowing through the flow path 227 is discharged through the opening portion 226. In this way, the flow path 227 is positioned downstream of the sweat collecting unit 21.

The first sensing unit 22 includes an in-mixed-liquid concentration measuring unit 24. The in-mixed-liquid concentration measuring unit 24 measures the concentration of the component to be measured in the mixed liquid using a predetermined measuring method. For example, the in-mixed-liquid concentration measuring unit 24 measures a value of a physical quantity which changes in accordance with a change in concentration of the component to be measured in the mixed liquid and converts the measurement result into the concentration in the mixed liquid, thereby measuring the concentration in the mixed liquid. Hereinafter, the concentration of the component to be measured in the mixed liquid measured by the in-mixed-liquid concentration measuring unit 24 is referred to as a first measurement value.

The in-mixed-liquid concentration measuring unit 24 outputs the first measurement value of the measurement result to the information processing unit 30 in a wireless or wired manner.

The in-mixed-liquid concentration measuring unit 24 may be any unit as long as it can acquire the first measurement value. One example of the in-mixed-liquid concentration measuring unit 24 will be described later.

The second sensing unit 23 includes a sheet-like fourth adhesive layer 231 and a sheet-like third dielectric layer 232 laminated in this order in the forward direction of the Z axis.

In the fourth adhesive layer 231, the opening portion 233 having a size in the XY plane larger than that of the opening portion 226 is formed. The opening portion 233 communicates with the opening portion 226.

The fourth adhesive layer 231 adheres the second dielectric layer 223 to the third dielectric layer 232. The fourth adhesive layer 231 may be any type as long as it is in the form of a sheet which adheres the second dielectric layer 223 to the third dielectric layer 232. The fourth adhesive layer 231 is, for example, a double-sided tape. A thickness (a length in the Z-axis direction) of the fourth adhesive layer 231 can be preferably 100 to 1000 μm and more preferably 500 μm.

In the third dielectric layer 232, the opening portion 234 having a size in the XY plane smaller than that of the opening portion 233 is formed. The opening portion 234 communicates with the opening portion 233. A line passing through a center of the opening portion 226 in the XY plane and parallel to the Z axis does not intersect with a line passing through a center of the opening portion 234 in the XY plane and parallel to the Z axis.

The third dielectric layer 232 is formed of a dielectric. The third dielectric layer 232 may be any type as long as it is a sheet-like dielectric. The third dielectric layer 232 may be, for example, a silicon sheet. A thickness (a length in the Z-axis direction) of the third dielectric layer 232 can be preferably 100 to 1000 μm and more preferably 500 μm.

A flow path 235 is formed using the opening portion 233 and the opening portion 234. The mixed liquid which has flowed through the flow path 227 flows into the second sensing unit 23 through the opening portion 226. The mixed liquid which has flowed into the opening portion 226 flows through the flow path 235. The mixed liquid flowing through the flow path 235 is discharged outside of the supplied unit 20 through the opening portion 234. In this way, the flow path 235 is positioned downstream of the sweat collecting unit 21.

The second sensing unit 23 includes a mixed liquid amount measurement unit 25. The mixed liquid amount measurement unit 25 measures an amount of the mixed liquid flowing through the flow path 235 using a predetermined measuring method. Hereinafter, an amount of the mixed liquid measured by the mixed liquid amount measurement unit 25 is referred to as a second measurement value.

The mixed liquid amount measurement unit 25 outputs the second measurement value of the measurement result to the information processing unit 30 in a wireless or wired manner.

The mixed liquid amount measurement unit 25 may be any unit as long as it can acquire an amount of the mixed liquid.

The mixed liquid amount measurement unit 25 may be, for example, a device which includes a central processing unit (CPU), a memory, an auxiliary storage device, and a light emitting diode (LED) and executes a first sweat amount measurement program. The mixed liquid amount measurement unit 25 receives absorbed, reflected, or scattering light of light emitted by the LED by executing an sweat amount measurement program and measures a time at which the mixed liquid flowing through the flow path 235 passes through a predetermined position on the basis of the light reception result. The mixed liquid amount measurement unit 25 measures the time at which the mixed liquid passes through the predetermined position to measure the second measurement value.

The mixed liquid amount measurement unit 25 may be, for example, a device which includes a CPU, a memory, an auxiliary storage device, and an imaging device and executes a second sweat amount measurement program. For example, when a scale is engraved in a third flow path 230, the mixed liquid amount measurement unit 25 may read a value of the scale in which a fluid is positioned using an imaging device by executing the second sweat amount measurement program to measure the second measurement value.

A description will be provided with reference to FIG. 1 again.

The information processing unit 30 functions as a device which includes an input unit 301, a control unit 302, a component concentration calculation unit 303, and an output unit 304.

The input unit 301 is constituted to include input devices such as a touch panel and a switch. The input unit 301 may be constituted as an interface configured to connect these input devices to a subject itself.

The input unit 301 receives processing start information concerning the subject itself as an input. If a start instruction is input to the input unit 301, the sweat measuring device 1 starts measuring the concentration of the component to be measured in sweat.

The control unit 302 controls an operation of the fluid supply unit 10, an operation of the in-mixed-liquid concentration measuring unit 24, and an operation of the mixed liquid amount measurement unit 25.

To be specific, the control unit 302 performs a cleaning control process, a measurement fluid control process, a first measurement value measurement control process, and a second measurement value measurement control process if a start condition is satisfied.

The start condition may be any condition as long as it associates with an input and a time of a start instruction to the input unit 301. The start condition may be, for example, a condition in which a start instruction is input to the input unit 301. The start condition may be, for example, a condition in which at least one of a first condition and a second condition is satisfied. The first condition is a condition in which a start instruction is input to the input unit 301. The second condition is a condition in which the measurement using the sweat measuring device 1 has been completed and then a predetermined time elapses. In such a case, the sweat measuring device 1 performs the measurement once at a timing at which the start instruction is input and then performs the measurement at a constant cycle.

The cleaning control process is a process of the control unit 302 which causes the fluid supply unit 10 to supply the auxiliary liquid to the supplied unit 20. The fluid supply unit 10 supplies the auxiliary liquid to the supplied unit 20 by executing the cleaning control process using the control unit 302.

The measurement fluid control process is a process executed by the control unit 302 after the cleaning control process ends and then a predetermined time (hereinafter referred to as an “inter-process time”) has elapsed. The measurement fluid control process is a process of the control unit 302 which causes the fluid supply unit 10 to supply a predetermined amount (hereinafter referred to as an “amount of auxiliary liquid”) of the auxiliary liquid to the supplied unit 20. The amount of the auxiliary liquid is, for example, an amount stored in an auxiliary storage device (not shown) included in the information processing unit 30. The fluid supply unit 10 supplies a predetermined amount of the auxiliary liquid to the supplied unit 20 by executing the measurement fluid control process using the control unit 302.

The first measurement value measurement control process is a process of the control unit 302 which causes the in-mixed-liquid concentration measuring unit 24 to measure the first measurement value. The in-mixed-liquid concentration measuring unit 24 acquires the first measurement value by executing the first measurement value measurement control process using the control unit 302.

The second measurement value measurement control process is a process of the control unit 302 which causes the mixed liquid amount measurement unit 25 to measure the second measurement value. The mixed liquid amount measurement unit 25 acquires the second measurement value by executing the second measurement value measurement control process using the control unit 302.

The component concentration calculation unit 303 calculates the concentration of the component to be measured in sweat by performing a component concentration calculation process.

The component concentration calculation unit 303 calculates the concentration of the component to be measured in sweat on the basis of the first measurement value, the second measurement value, and the amount of the auxiliary liquid by performing the component concentration calculation process.

The output unit 304 outputs the result calculated by the component concentration calculation unit 303. The output unit 304 may be constituted to include display devices such as a cathode ray tube (CRT) display, a liquid crystal display, and an organic electro-luminescence (EL) display. The output unit 304 may be constituted as an interface configured to connect these display devices to the subject itself.

(Example of in-Mixed-Liquid Concentration Measuring Unit 24)

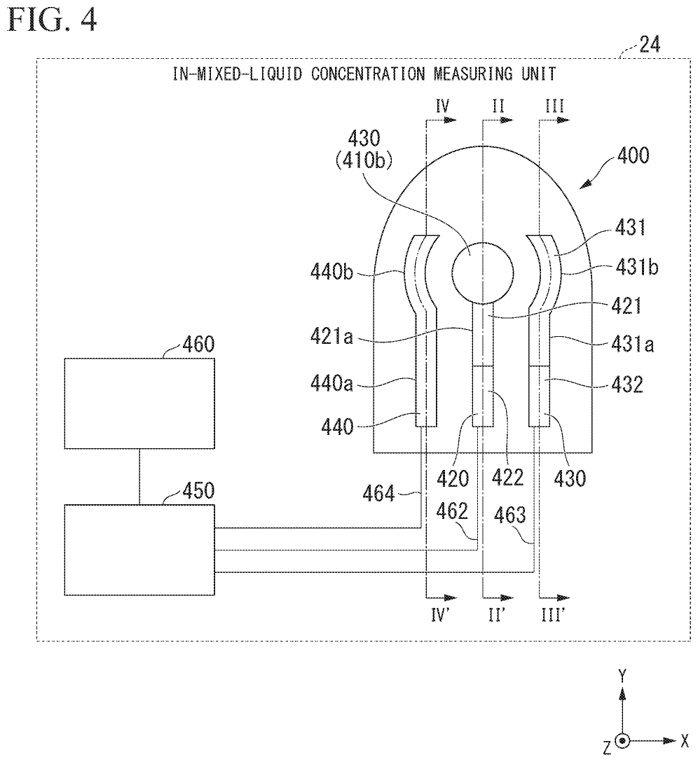

FIG. 4 is a diagram showing an example of a constitution of the in-mixed-liquid concentration measuring unit 24 in the embodiment.

The in-mixed-liquid concentration measuring unit 24 includes a biological information measurement sensor 400, a potentiostat 450, and a current value concentration conversion unit 460.

### (Description of Biological Information Measurement Sensor 400)

First, the biological information measurement sensor 400 will be described.

The biological information measurement sensor 400 includes an insulating base member 410, and a working electrode 420, a counter electrode 430, and a reference electrode 440 above the insulating base member 410.

The counter electrode 430 and the reference electrode 440 are provided above the insulating base member 410 so that they are disposed on both sides of the working electrode 420.

The insulating base member 410 is constituted of an insulating material so that three electrodes (that is, three electrodes, i.e., the working electrode 420, the counter electrode 430, and the reference electrode 440) are not short-circuited.

The insulating base member 410 can be preferably constituted of a flexible material so that the insulating base member 410 easily comes into close contact with a living body. Furthermore, the insulating base member 410 is required to have a strength in which the three electrodes can be held.

Examples of base materials in which these conditions are satisfied include polyimide films, polyesters, polymer films such as polytetrafluoroethylene (PTFE), paper, and ceramics such as mica.

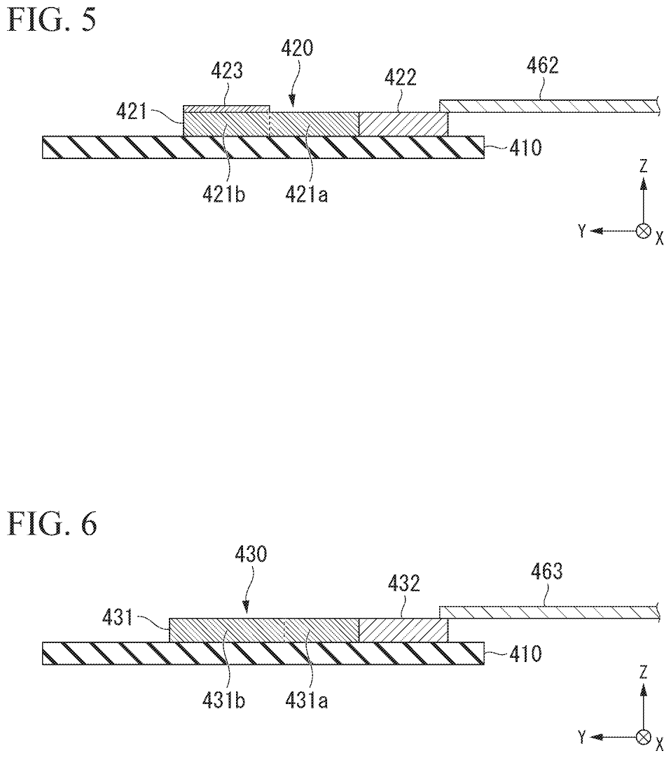

The working electrode 420 is an electrode which exchanges electrons with the component to be measured. In this embodiment, as shown in FIGS. 4 and 5, the working electrode 420 is constituted of a working electrode main body 421, a working electrode connection section 422 continuous with the working electrode main body 421, and an enzyme film 423 provided above the working electrode main body 421. FIG. 5 is a diagram showing the working electrode 420 when viewed from a direction perpendicular to that of FIG. 4.

The working electrode main body 421 is made of a conductive solid material such as, for example, carbon, platinum, and gold. The working electrode main body 421 in this embodiment includes a straight linear section 421a and a circular section 421b integrally formed with a first end portion of the straight linear section 421a. Furthermore, the working electrode connection section 422 is coaxially connected to a second end portion of the straight linear section 421a. The working electrode connection section 422 is made of, for example, a metal having a low resistance value such as silver/silver chloride, silver, copper, platinum, and gold.

The enzyme film 423 is provided above the circular section 421b of the working electrode main body 421. Examples of the enzyme film 423 include an enzyme film made of a material selected from the group consisting of lactate oxidase and glucose oxidase.

When the enzyme film 423 is lactate oxidase, a reaction for converting lactic acid to pyruvic acid occurs in the enzyme film 423 and hydrogen peroxide generated at the time of the reaction reacts with a dye (Prussian blue) in the electrode to generate a current.

The counter electrode 430 is an electrode in which a current flows between the counter electrode 430 and the working electrode 420. In this embodiment, as shown in FIGS. 4 and 6, the counter electrode 430 is constituted of a counter electrode main body 431 and a counter electrode connection section 432 continuous with the counter electrode main body 431. FIG. 6 is a diagram showing the counter electrode 430 when viewed from a direction perpendicular to that of FIG. 4.

The counter electrode main body 431 is made of a conductive solid material such as, for example, carbon, platinum, and gold. The counter electrode main body 431 in this embodiment includes a straight linear section 431a and a circular arc section 431b integrally formed with a first end portion of the straight linear section 431a. The circular arc section 431b is formed in a circular arc shape to surround the circular section 421b of the working electrode main body 421 at substantially the same distance from the circular section 421b. Furthermore, the counter electrode connection section 432 is coaxially connected to a second end portion of the straight linear section 431a. The counter electrode connection section 432 is made of, for example, a metal having a low resistance value such as silver/silver chloride, silver, copper, platinum, and gold.

The reference electrode 440 is an electrode serving as a reference for a potential of the working electrode 420. The reference electrode 440 is made of silver/silver chloride. As shown in FIGS. 4 and 6, the reference electrode 440 in this embodiment includes a straight linear section 440a and a circular arc section 440b integrally formed with a first end portion of the straight linear section 440a. The circular arc section 440b is formed in a circular arc shape to surround the circular section 421b of the working electrode main body 421 at substantially the same distance from the circular section 421b together with the circular arc section 431b of the counter electrode 430. FIG. 6 is a diagram showing the reference electrode 440 when viewed from a direction perpendicular to that of FIG. 4.

Thicknesses of the working electrode main body 421, the counter electrode main body 431, and the reference electrode 440 can be preferably 10 to 100 μm.

When the thicknesses of these members are equal to or more than preferred lower limit values, it is possible to stabilize an output of the biological information measurement sensor 400. Furthermore, the thicknesses of these members are equal to or less than preferred upper limit values, it is easy to secure the flexibility of the biological information measurement sensor 400 and the biological information measurement sensor 400 easily follows the movement of a living body.

Also, widths of the straight linear section 421a of the working electrode main body 421, the counter electrode main body 431, and the reference electrode 440 can be preferably 0.05 to 3 mm and more preferably 0.3 to 1.5 mm.

When the widths of these members are equal to or more than preferred lower limit values, it is easy to stabilize an output of the biological information measurement sensor 400. Furthermore, when the widths of these members are equal to or less than preferred upper limit values, it is possible to form the entire biological information measurement sensor 400 to have a smaller size.

An amount of enzyme used for forming the enzyme film 423 can be preferably 3.2 to 25.6 units and more preferably 12.8 units.

If the amount of enzyme used for forming the enzyme film 423 is equal to or more than a preferred lower limit value, a sufficient sensitivity can be obtained. If the amount of enzyme used for forming the enzyme film 423 is equal to or less than a preferred upper limit value, the costs can be reduced.

An area of the circular section 421b in the working electrode main body 421 can be preferably 1 to 30 mm² and more preferably 1.5 to 20 mm². If the area of the circular section 421b is equal to or more than a preferred lower limit value, it is easy to form the enzyme film 423 with a sufficient amount of enzyme. If the area of the circular section 421b is equal to or less than a preferred upper limit value, it is possible to form the entire biological information measurement sensor 400 to have a smaller size.

A separation distance between the working electrode 420 and the counter electrode 430 and a separation distance between the working electrode 420 and the reference electrode 440 can be preferably 0.5 to 3 mm and more preferably 1 to 2 mm.

When the separation distances are equal to or more than preferred lower limit values, an excellent production efficiency is provided at the time of producing electrodes. When the separation distances are equal to or less than preferred upper limit values, it is possible to accurately perform the measurement even if an amount of specimen is reduced at the time of performing the measurement.

### (Description of Potentiostat 450)

The potentiostat 450 will be described below.

The potentiostat 450 is connected to the biological information measurement sensor 400 using a working electrode lead wire 462, a counter electrode lead wire 463, and a reference electrode lead wire 464.

The working electrode lead wire 462 is a lead wire configured to connect the working electrode 420 to the potentiostat 450. The counter electrode lead wire 463 is a lead wire configured to connect the counter electrode 430 to the potentiostat 450. The reference electrode lead wire 464 is a lead wire configured to connect the reference electrode 440 to the potentiostat 450.

The potentiostat 450 applies a constant potential with respect to the reference electrode 440 to the working electrode 420 and measures a current between the working electrode 420 and the counter electrode 430.

### (Description of Current Value Concentration Conversion Unit 460)

The current value concentration conversion unit 460 acquires the concentration of the component to be measured in the mixed liquid on the basis of a current value of a current measured by the potentiostat 450.

### (Description of Operations of Biological Information Measurement Sensor 400, Potentiostat 450, and Current Value Concentration Conversion Unit 460)

The potentiostat 450 applies a predetermined constant potential with respect to the reference electrode 440 to the working electrode 420 of the biological information measurement sensor 400. Furthermore, a current (a current flowing between the working electrode 420 and the counter electrode 430) obtained from the biological information measurement sensor 400 at this time is detected using the potentiostat 450.

When the biological information measurement sensor 400 is positioned in the mixed liquid, a current flowing between the working electrode 420 and the counter electrode 430 is a current of a current value according to the concentration of the component to be measured in the mixed liquid. For this reason, when the biological information measurement sensor 400 is positioned in the mixed liquid, a current value of a current measured by the potentiostat 450 is a value of a physical quantity which changes in accordance with a change in concentration of the component to be measured in the mixed liquid.

The current value concentration conversion unit 460 acquires the first measurement value which is the concentration of the component to be measured in the mixed liquid on the basis of the current value of the current measured by the potentiostat 450.

### (Top View of Sweat Collecting Unit 21 and First Sensing Unit 22)

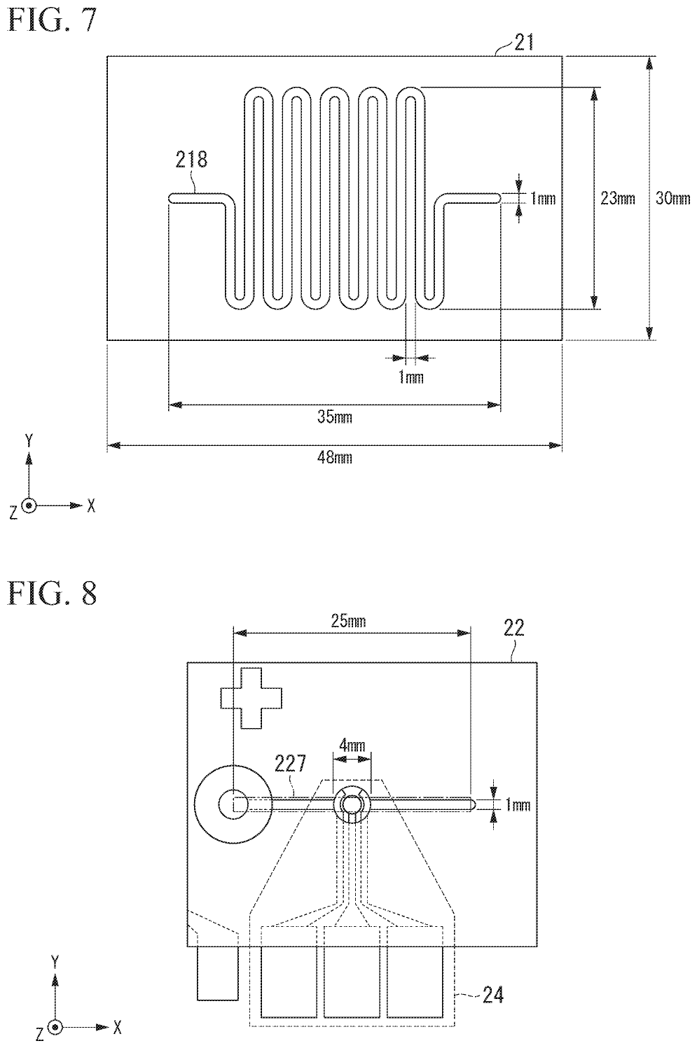

FIG. 7 is a top view of the sweat collecting unit 21 in the embodiment. FIG. 7 shows the sweat collecting unit 21 having a size of 30 mm×48 mm FIG. 7 shows the flow path 218 formed in a bellows shape. FIG. 7 shows the flow path 218 having a size and a shape in which the flow path 218 is positioned in a rectangular shape of 23 mm×35 mm FIG. 7 shows the flow path 218 having a width of a flow path thereof being 1 mm FIG. 7 shows the flow path 218 having a distance between flow paths thereof being 1 mm.

FIG. 8 is a top view of the first sensing unit 22 in the embodiment. FIG. 8 shows the flow path 227 having a straight linear shape with a length of 25 mm and a width being 1 mm FIG. 8 is a top view of the in-mixed-liquid concentration measuring unit 24. FIG. 8 shows a part of the in-mixed-liquid concentration measuring unit 24 positioned in a region with a length of 4 mm in a center of the flow path 227.

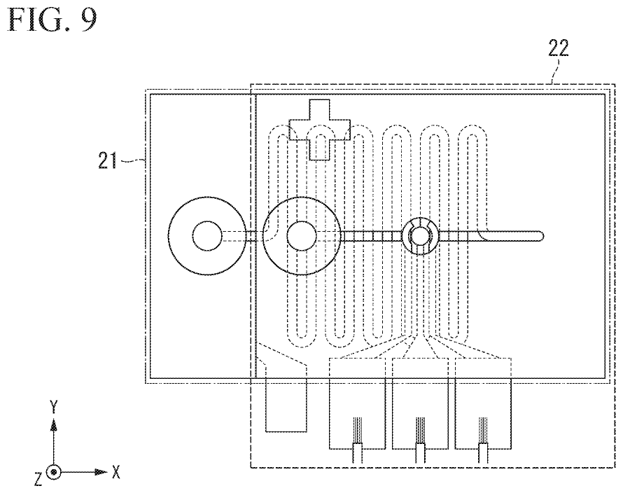

FIG. 9 is a top view of the sweat collecting unit 21 and the first sensing unit 22 which have been laminated in the embodiment. FIG. 9 shows the first sensing unit 22 laminated on the sweat collecting unit 21 and one end portion of the flow path 218 and one end portion of the flow path 227 overlapping. In FIG. 9, one end portion of the flow path 218 overlapping one end portion of the flow path 227 is an example of the opening portion 217. In FIG. 9, one end portion of the flow path 227 overlapping one end portion of the flow path 218 is an example of the opening portion 224.

### (Flowchart)

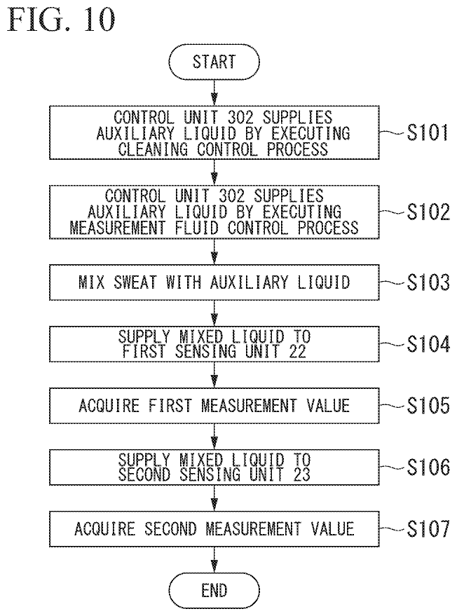

FIG. 10 is a flowchart for describing a flow of a process from when a start condition is satisfied to when the sweat measuring device 1 calculates the concentration of a component to be measured in sweat in the embodiment.

The control unit 302 executes the cleaning control process and the fluid supply unit 10 supplies the auxiliary liquid to the supplied unit 20 (Step S101). To be specific, the fluid supply unit 10 supplies the auxiliary liquid to the supplied unit 20 by being controlled through the cleaning control process executed by the control unit 302. The auxiliary liquid supplied to the supplied unit 20 mixes with old sweat accumulated in the supplied unit 20 and is discharged to the outside of the supplied unit 20 through the opening portion 234. For this reason, the supplied unit 20 can remove old sweat accumulated in the supplied unit 20 through the process of Step S101.

After the cleaning control process is completed and then the inter-process time elapses, the control unit 302 executes the measurement fluid control process. When the control unit 302 executes the measurement fluid control process, the fluid supply unit 10 supplies the auxiliary liquid of the amount of the auxiliary liquid to the supplied unit 20 (Step S102). To be specific, the fluid supply unit 10 supplies the auxiliary liquid of the amount of the auxiliary liquid to the supplied unit 20 by being controlled through the measurement fluid control process executed by the control unit 302.

In the sweat collecting unit 21, the auxiliary liquid flowing through the flow path 218 in the sweat collecting unit 21 mixes with sweat discharged into the sweat collecting unit 21 (Step S103). In Step S103, the sweat mixing with the auxiliary liquid is sweat which has flowed from the skin 9 into the supplied unit 20 during the inter-process time.

The mixed liquid which is a liquid obtained by mixing the auxiliary liquid with the sweat is supplied from the sweat collecting unit 21 to the first sensing unit 22 (Step S104). To be specific, when the mixed liquid flows from the flow path 218 to the flow path 227, the mixed liquid is supplied from the sweat collecting unit 21 to the first sensing unit 22.

In the first sensing unit 22, the first measurement value is acquired (Step S105). To be specific, the control unit 302 executes the first measurement value measurement control process and the in-mixed-liquid concentration measuring unit 24 measures the concentration of the component to be measured in the mixed liquid flowing through the flow path 227. An operation of the in-mixed-liquid concentration measuring unit 24 in Step S105 is controlled through the first measurement value measurement control process executed by the control unit 302.

The mixed liquid is supplied from the first sensing unit 22 to the second sensing unit 23 (Step S106). To be specific, when the mixed liquid flows from the flow path 227 to the flow path 235, the mixed liquid is supplied from the first sensing unit 22 to the second sensing unit 23.

In the second sensing unit 23, the second measurement value is acquired (Step S107). To be specific, the control unit 302 executes the second measurement value measurement control process and the mixed liquid amount measurement unit 25 measures an amount of the mixed liquid. An operation of the mixed liquid amount measurement unit 25 in Step S107 is controlled through the second measurement value measurement control process executed by the control unit 302.

The sweat measuring device 1 in the embodiment constituted as described above includes the fluid supply unit 10 and the supplied unit 20 and the cleaning control process is executed before the measurement fluid control process. Thus, it is possible to measure the concentration of the component to be measured by reducing an influence of old sweat. For this reason, the sweat measuring device 1 can prevent a decrease in measurement accuracy of an amount of component in a liquid (sweat) discharged from sweat which is a discharge source.

Also, the sweat measuring device 1 in the embodiment constituted as described above measures the concentration of the component to be measured using not only the sweat but also the auxiliary liquid. For this reason, the sweat measuring device 1 prevents an increase in time required for collecting the sweat and the measurement using the sweat measuring device 1 does not impair the real-time property. For this reason, the sweat measuring device 1 can prevent a decrease in measurement accuracy due to the time required for collecting the sweat.

Also, the sweat measuring device 1 in the embodiment constituted as described above includes the sweat collecting unit 21, the first sensing unit 22, and the second sensing unit 23 laminated in the direction perpendicular to the surface of the skin 9. For this reason, the sweat measuring device 1 can prevent an increase in area of a device and prevent an increase in size of a device.

Furthermore, the sweat measuring device 1 in the embodiment constituted as described above measures the concentration of the component to be measured using not only the sweat but also the auxiliary liquid. For this reason, the sweat measuring device 1 can measure the concentration of the component to be measured using sweat less than that of a case in which the measurement is performed without using the auxiliary liquid. For this reason, an area of the sweat collecting unit 21 in contact with the skin 9 is smaller than that a case in which the measurement is performed without using the auxiliary liquid. For this reason, the sweat measuring device 1 can prevent an increase in area of a device and prevent an increase in size of a device.

### (Modification)

The working electrode 420 is not limited to the electrode having the enzyme film. For example, the working electrode 420 may be an electrode obtained by adhering an oxygen permeable film to a surface of a conductive solid material.

Also, the working electrode lead wire 462 may be directly connected to a main body of the working electrode 420 made of carbon or the like without passing through the working electrode connection section. Similarly, the counter electrode lead wire 463 may be directly connected to a main body of the counter electrode 430 made of carbon or the like without passing through the counter electrode connection section.

There is no particular limitation on a shape of each of the three electrodes and a mutual positional relationship among these three electrodes. It is also possible to appropriately change a shape of the insulating base member to correspond to the shape of each of the three electrodes the mutual positional relationship among these three electrodes.

Also, the potentiostat 450 and the biological information measurement sensor 400 may be connected wirelessly without using a lead wire. In the case of wireless connection, for example, it is possible to utilize short-range wireless communication such as Bluetooth (registered trademark).

The sweat collecting unit 21, the first sensing unit 22, and the second sensing unit 23 do not necessarily need to be all laminated. Only the sweat collecting unit 21 and the first sensing unit 22 may be laminated and only the sweat collecting unit 21 and the second sensing unit 23 may be laminated. Only the first sensing unit 22 and the second sensing unit 23 may be laminated. In this way, the sweat collecting unit 21, the first sensing unit 22, and the second sensing unit 23 do not all need to be laminated and at least two thereof may be laminated.

It is not always necessary that the sweat collecting unit 21 is connected to the first sensing unit 22 and the first sensing unit 22 is connected to the second sensing unit 23. In the sweat collecting unit 21, the first sensing unit 22, and the second sensing unit 23, if the sweat collecting unit 21 is in contact with the skin 9, the sweat collecting unit 21 may be connected to the second sensing unit 23 and the second sensing unit 23 may be connected to the first sensing unit 22.

Sweat is an example of a main liquid and the skin 9 is an example of a discharge source. The main liquid may be a sap. In this case, the discharge source may be the bark of a tree. The flow path 227 is an example of a first flow path. The flow path 235 is an example of a second flow path. The sweat collecting unit 21 is an example of a liquid collecting unit. The sweat measuring device 1 is an example of a component concentration measuring device. The first measurement value is an example of a first measurement target component concentration. The second measurement value is an example of an amount of mixed liquid. The concentration of the component to be measured in sweat is an example of a second measurement target component concentration. The fluid supply unit 10 is an example of an auxiliary fluid supply unit.

All or some of functions of the sweat measuring device 1 may be realized using hardware such as an application specific integrated circuit (ASIC), a programmable logic device (PLD), and a field programmable gate array (FPGA). The program may be recorded on a computer-readable recording medium. The computer-readable recording medium is, for example, a portable medium such as a flexible disk, a magneto-optical disk, a read only memory (ROM), a compact disk (CD)-ROM, or a storage device such as a hard disk built in a computer system. The program may be transmitted via a telecommunication line.

The sweat measuring device 1 may be implemented using multiple information processing devices communicably connected over a network. In this case, functional units included in the sweat measuring device 1 may be separately implemented in the multiple information processing devices. For example, the information processing unit 30 and the other functional units may be implemented in different information processing devices.

Although the embodiments of the present invention have been described in detail above with reference to the drawings, the specific constitution is not limited to the embodiments and includes a design and the like without departing from the scope of the present invention.

What is claimed is:

1. A component concentration measuring device, comprising:
a liquid collecting unit configured to collect a main liquid including a measurement target component to be measured and discharge a mixed liquid obtained by mixing the main liquid with an auxiliary liquid supplied from an outside; and
a first sensing unit comprising a first flow path positioned downstream of the liquid collecting unit and an in-mixed-liquid concentration measuring unit positioned in the first flow path and configured to measure a first measurement target component concentration which is a concentration of the measurement target component in the mixed liquid.

2. The component concentration measuring device according to claim 1, comprising:
a second sensing unit comprising a second flow path positioned downstream of the liquid collecting unit and a mixed liquid amount measurement unit configured to measure a mixed liquid amount which is an amount of the mixed liquid flowing through the second flow path.

3. The component concentration measuring device according to claim 2, further comprising:
a component concentration calculation unit configured to calculate a second measurement target component concentration which is a concentration of the measurement target component in the main liquid based on the first measurement target component concentration, the mixed liquid amount, and an auxiliary liquid amount which is an amount of the auxiliary liquid supplied from the outside.

4. The component concentration measuring device according to claim 1, further comprising:
an auxiliary fluid supply unit configured to supply the auxiliary liquid to the liquid collecting unit,
wherein the auxiliary fluid supply unit is configured to supply the auxiliary liquid to the first flow path after the auxiliary liquid has been supplied to the first flow path and then an inter-process time which is a predetermined time has elapsed, and
the in-mixed-liquid concentration measuring unit is configured to measure a concentration of the measurement target component in the mixed liquid including the auxiliary liquid supplied to the first flow path by the auxiliary fluid supply unit after the inter-process time has elapsed.

5. The component concentration measuring device according to claim 1,
wherein the liquid collecting unit and the first sensing unit are laminated.

6. The component concentration measuring device according to claim 2,
wherein at least two of the liquid collecting unit, the first sensing unit, and the second sensing unit are laminated.

7. A component concentration measuring method, comprising:
collecting a main liquid including a measurement target component to be measured and discharging a mixed liquid obtained by mixing the main liquid with an auxiliary liquid supplied from an outside; and
measuring a first measurement target component concentration which is a concentration of the measurement target component in the mixed liquid.
